(19) United States
(12) Patent Application Publication
(10) Pub. No.: US 2020/0163882 A1
(43) Pub. Date: May 28, 2020

(54) Solid Pharmaceutical Compositions Of Androgen Receptor Antagonists

(71) Applicant: Lek Pharmaceuticals d.d., Ljubljana (SI)

(72) Inventors: Rok Grahek, Ljubljana (SI); Andrija Lebar, Ljubljana (SI); Petra Draksler, Ljubljana (SI); Bostjan Petek, Ljubljana (SI); Jerneja Opara, Ljubljana (SI); Klemen Naversnik, Ljubljana (SI); Petra Bozic, Ljubljana (SI)

(73) Assignee: Lek Pharmaceuticals d.d., Ljubljana (SI)

(21) Appl. No.: 16/751,521

(22) Filed: Jan. 24, 2020

Related U.S. Application Data
(63) Continuation of application No. 15/114,890, filed on Jul. 28, 2016.

(30) Foreign Application Priority Data
Feb. 5, 2014 (EP) 14154047.6

(51) Int. Cl. A61K 9/14; A61K 31/4166; A61K 9/48; A61K 31/4439; A61K 9/20; A61K 31/4152; A61K 9/16

(52) U.S. Cl. CPC A61K 9/143; A61K 9/1623; A61K 9/485; A61K 9/4825; A61K 31/4439; A61K 9/2009; A61K 31/4152; A61K 9/2013; A61K 9/2018; A61K 9/1641; A61K 9/1652; A61K 9/1611; A61K 9/1617; A61K 9/2054; A61K 9/2031; A61K 31/4166

(57) ABSTRACT

The present invention belongs to the field of pharmaceutical industry and relates to a solid pharmaceutical composition comprising androgen receptor antagonists, e.g. Enzalutamide or ARN-509, as well as to processes for preparing the same. The solid pharmaceutical compositions are useful in the treatment of prostate cancer.

This application is a continuation of co-pending application Ser. No. 15/114,890, having a Section 371 date of Jul. 28, 2016, which in turn is a Section 371 national phase entry of PCT application PCT/EP2015/052311, filed Feb. 4, 2015. This application also claims the benefit of the earlier filing date of European patent application 14154047.6, filed Feb. 5, 2014.

### FIELD OF THE INVENTION

The present invention belongs to the field of pharmaceutical industry and relates to solid pharmaceutical compositions of androgen receptor antagonists, as well as to processes for preparing the same. Such solid pharmaceutical compositions are useful in the treatment of prostate cancer.

### DESCRIPTION OF THE BACKGROUND ART

Enzalutamide (chemical name: 4-{3-[4-Cyano-3-(trifluoromethyl)phenyl]-5,5-dimethyl-4-oxo-2-thioxoimidazolidin-1-yl}-2-fluoro-N-methylbenzamide) and ARN-509 (chemical name: 4-[7-[6-Cyano-5-(trifluoromethyl)pyridin-3-yl]-8-oxo-6-thioxo-5,7-diazaspiro[3.4]octan-5-yl]-2-fluoro-N-methylbenzamide) are androgen receptor antagonists indicated for the treatment of male patients with metastatic castration-resistant prostate cancer. The structures of both these API, which are shown below, are closely related:

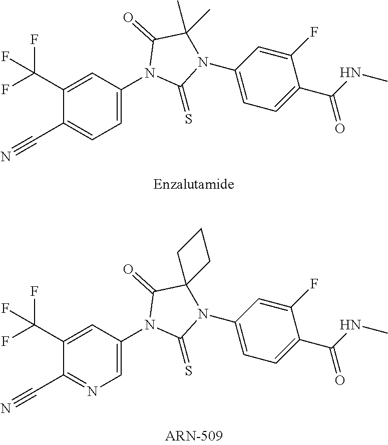 [SMILES: CNC(=O)c1ccc(N2C(=S)N(c3ccc(C#N)c(C(F)(F)F)c3)C(=O)C2(C)C)cc1F ; CNC(=O)c1ccc(N2C(=S)N(c3cnc(C#N)c(C(F)(F)F)c3)C(=O)C23CCC3)cc1F]

Only general disclosures of formulations of Enzalutamide are present in WO2006/124118A1, which discloses its preparation in example 56 (Enzalutamide then called RD162′) and which generically describes pharmaceutical compositions and dosages.

Disclosures of formulations of ARN-509, but again in a general way, are presented in WO2007/126765A1, which discloses its preparation in par. [0055] (ARN-509 was then called A52). Pharmaceutical compositions and dosages are generically described, including an exemplified oral test formulation in the form of a liquid, DMSO-containing suspension. Due to high DMSO content and instable suspension, such a test formulation is unsuitable for pharmaceutical use.

WO 2013/184681 A1 is directed to crystal forms of ARN-509 and discloses a capsule containing the pure crystalline API (page 43).

Enzalutamide and ARN-509 are poorly soluble; in particular they are sparingly soluble in absolute ethanol and practically insoluble in water between pH 1 and 11. They are soluble in acetone and N-methyl-2-pyrrolidone (NMP). Further, they are non-hygroscopic, crystalline solids that remain unionized over the physiologic pH range. They belong to Class 2 drugs using the Biopharmaceutics Classification System. Poor drug solubility however represents a bottleneck for dissolution, which in turn critically affects drug bioavailability.

Owing to the afore-described constraints in dissolution and bioavailability, the currently marketed formulation of Enzalutamide (Xtandi®) contains 40 mg of Enzalutamide as a solution in a mixture of caprylocaproyl polyoxylglycerides (Labrasol®), antioxidants butylated hydroxyanisole (BHA) and butylated hydroxytoluene (BHT) inside a soft gelatin capsule. Other inactive ingredients are gelatin, sorbitol sorbitan solution, glycerin, purified water, titanium dioxide and black iron oxide. Because of all the inactive ingredients the soft gelatin capsules are very big (weight 1460 mg, volume about 1.3 cm³).

With such formulation a dissolution step is completely by-passed in vivo, as upon administration Enzalutamide enters the gastro-intestinal tract already dissolved in caprylocaproyl polyoxylglycerides (Labrasol®).

The recommended dose is 160 mg given once daily, which represents four capsules, each containing 40 mg of Enzalutamide. The patient should swallow the whole capsule which should not be chewed, dissolved or opened prior to swallowing, because Enzalutamide itself represents a risk for the patient or other persons in contact with the capsule if the capsule is opened and the liquid comes out.

Patient compliance of Xtandi is therefore problematic for a number of reasons. The patient has to swallow multiple capsules of considerable size, and ensure that no damage to the capsules and thus consequent leakage occurs before they reach the gastro-intestinal tract. This represents in particular difficulty for (mostly elderly) patients suffering from the disease and side effects of the therapy itself.

Another patient safety concern arises from extremely high content of surface active substances in the currently marketed Enzalutamide formulation. Taking one daily recommended dose (160 mg) results in digesting about 3600 mg of caprylocaproyl polyoxylglycerides (Labrasol®), which exceeds over 50-times the FDA's Inactive Ingredient Guide's (IIG; status October 2013) daily limit of 70 mg/day. In addition, Xtandi comprises two antioxidants, butylated hydroxyanisole (BHA) and butylated hydroxytoluene (BHT). The quantity of BHA in one recommended daily dose is about 3.7 mg and significantly exceeds the IIG daily limit of 1 mg/day. The quantity of BHT in recommended daily dose is about 0.22 mg and is on par with IIG daily limit of 0.2 mg/day for soft gelatin capsule. All these ingredients represent an enormous bio-burden for the patient during the therapy, adding up to the burden of the disease and the side effects of Enzalutamide itself.

ARN-509 is a molecule that is very similar to Enzalutamide. While physical properties, such as dissolution, are similar to those of Enzalutamide, first clinical trials suggest that this molecule is more effective than Enzalutamide at similar daily doses.

There is thus a need, and hence it is an object of the present invention, to provide compositions or formulations of Enzalutamide and ARN-509 and closely related androgen receptor antagonists with improved pharmaceutical attributes including relatively fast dissolution. Further desirable objects which shall be achievable as further improved and preferred pharmaceutical attributes include, alone and preferably in combination:
ensure high bioavailability, which besides the basically obtained fast dissolution more preferably further attains relatively low level or slow rate of precipitation in bio-relevant media such as simulated gastric or intestinal fluid;
provide small dosage forms in weight and in physical volume in order to be easily swallowable by patients and come in a small number of units per daily recommended dose, preferably in single dosage unit, in order to enhance patient compliance;
offer protection of patient or other persons in contact with the dosage form against leakage on breaking or other physical contact with active ingredient, such as entirely solid formulations, e.g. tablets (preferably film coated) or capsules with solid content;
contain low content of surface active substances, antioxidants and other ingredients that significantly elevate bio-burden to patients undergoing drug therapy;
are chemically stable.

A further object is to provide processes by which compositions or formulations of such androgen receptor antagonists can be efficiently prepared by using common pharmaceutical technologies at relatively low costs, e.g. can be processed simply using mixing, granulation, tableting, pelletisation, capsulation, coating and similar.

These objects as well as other preferred objects, which will become apparent from the following description of the present invention, can be made possible by the subject-matter of the independent claims. Some of the preferred embodiments of the present invention are defined by the subject matter of the dependent claims.

### SUMMARY OF THE INVENTION

The present invention provides, as set forth in the following items, various aspects, subject-matters and preferred embodiments, which respectively taken alone or in combination, contribute to solving the object of the present invention as well as further objects:

(1) A solid pharmaceutical composition comprising

(a) a compound of formula I

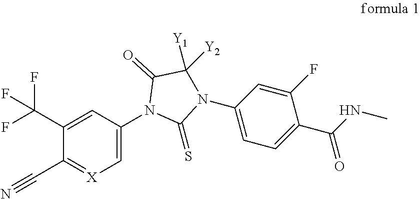

in which X is C or N, and Y₁ and Y₂ either denote CH₃ respectively, or Y₁ and Y₂ are interconnected to form a cyclobutane ring,

(b) a carrier, and

(c) a surfactant

wherein the compound of formula 1 is mainly amorphous.

(2) The solid pharmaceutical composition according to item (1), wherein the amount of surfactant is limited by a weight ratio of the surfactant to the compound of formula 1 being not higher than 10:1, preferably not higher than 5:1, more preferably not higher than 2:1.

(3) The solid pharmaceutical composition according to item (1) or (2), wherein the weight ratio of the surfactant to the compound of formula 1 lies in a range of 5:1 to 1:10, preferably 3:1 to 1:5, more preferably 2:1 to 1:2.

(4) The solid pharmaceutical composition according to anyone of items (1) to (3), wherein the amount of surfactant in the whole composition is at least 0.5 wt. %, provided that the defined weight ratio to the compound of formula 1 is met.

(5) The solid pharmaceutical composition according to anyone of the preceding items, having a dissolution ratio of the compound of formula 1 of not less than (NLT) 35%, when the pharmaceutical composition is subjected to a dissolution test in fasted state simulated intestinal fluid (FaSSIF) pH 6.5 medium at 45 minutes and at 100 rpm in USP Apparatus 2 (paddle method).

(6) The solid pharmaceutical composition according to item (5), having a dissolution ratio of the compound of formula 1 of not less than (NLT) 40% when subjected to said FaSSIF dissolution test.

(7) The solid pharmaceutical composition according to any one of the preceding items, wherein the amount of the compound of formula 1 in the entire composition is greater than 5%, preferably greater than 10%, more preferably greater than 15%.

(8) The solid pharmaceutical composition according to anyone of the preceding items, wherein the compound of formula 1 is substantially amorphous and preferably entirely amorphous.

(9) The solid pharmaceutical composition according to anyone of the preceding items, wherein the surfactant is selected from the group consisting of sodium lauryl sulphate; polyethylene glycol having molecular weight in the range of about 2000 to 10000; Polysorbates; fatty acid esters, preferably propylene glycol caprylates such as Capmul PG-8, Capryol 90; esters of glycerol and fatty acids, preferably glycerol oleates and caprylates (Capmul MCM); esters of polyethylene glycol and fatty acids, castor oil ethoxylate (glycerol polyethylene glycol ricinoleate).

(10) The solid pharmaceutical composition according to anyone of the preceding items, wherein the surfactant is selected from the group consisting of sodium lauryl sulphate; PEG 3350, PEG 4000, PEG 6000 or, PEG 8000, more preferably PEG 6000; Tween 20 or Tween 80; and esters of polyethylene glycol and fatty acids; most preferably sodium lauryl sulphate.

(11) The solid pharmaceutical composition according to any one of the preceding items, wherein the compound of formula 1 is Enzalutamide, thus wherein X═C and Y₁═Y₂═CH₃ denoted by the following formula:

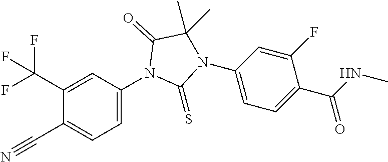 [SMILES: CNC(=O)c1ccc(N2C(=S)N(c3ccc(C#N)c(C(F)(F)F)c3)C(=O)C2(C)C)cc1F]

(12) The solid pharmaceutical composition according to any one of the preceding items, wherein the compound of formula 1 is ARN-509, thus wherein X═N and Y₁ and Y₂ being interconnected to form a cyclobutane ring, denoted by the following formula:

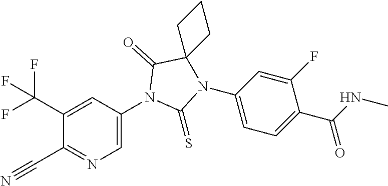 [SMILES: CNC(=O)c1ccc(N2C(=S)N(c3cnc(C#N)c(C(F)(F)F)c3)C(=O)C23CCC3)cc1F]

(13) The solid pharmaceutical composition according to any one of the preceding items, wherein the compound of formula 1 and the carrier are in association with each other, without separation therebetween.

(14) The solid pharmaceutical composition according to anyone of the preceding items, wherein components (a) and (b) are combined in the form of a solid adsorbate of said compound of formula 1 being adsorbed on the surface of the carrier.

(15) The solid pharmaceutical composition according to anyone of the preceding items, wherein the carrier is a particulate carrier having a BET-surface area of at least 10 m²/g, more preferably at least 50 m²/g, more preferably at least 250 m²/g.

(16) The solid pharmaceutical composition according to anyone of the preceding items, wherein the carrier is selected from the group consisting of alumosilicate and silicon dioxide, preferably selected from magnesium aluminometasilicate and colloidal silicon dioxide and porous silica, most preferably Syloid or Aerosil type silica or Neusilin.

(17) The solid pharmaceutical composition according to anyone of items (14) to (16), wherein the amount of the compound of formula 1 in the adsorbate is in the range of about 2 to about 35 wt.-%, preferably in the range of about 3 to about 30 wt.-%, more preferably in the range of about 5 to about 25 wt.-%, and even more preferably in the range of about 10 to about 20 wt.-%, respectively in % by weight relative to the whole adsorbate.

(18) The solid pharmaceutical composition according to anyone of items (1) to (13), wherein components (a) and (b) are combined in the form of a solid dispersion or a solid solution of said compound of formula 1 with a polymer.

(19) The solid pharmaceutical composition according to item (18), wherein the solid dispersion of the polymer and the compound of formula 1 is substantially homogeneous.

(20) The solid pharmaceutical composition according to item (18) or (19), wherein the carrier is formed by the polymer.

(21) The solid pharmaceutical composition according to anyone of items (18) to (20), wherein the solid dispersion is formed with a hydrophilic polymer, preferably the hydrophilic polymer is water soluble, more preferably said hydrophilic polymer is selected from cellulose derivatives, polyvinyl pyrrolidone (PVP) and polyvinyl alcohol (PVA).

(22) The solid pharmaceutical composition according to anyone of items (18) to (21), wherein said solid dispersion is formed with at least one polymer selected from the group consisting of hydroxyethyl cellulose (HEC), hydroxypropyl cellulose (HPC), hydroxypropyl methyl cellulose (HPMC), polyvinyl pyrrolidone (PVP), polyvinyl alcohol (PVA), polyacrylic acid (PAA), poly(ethylene glycol) (PEG), poly(ethylene oxide) (PEO), copovidone, hypromellose acetate succinate (HPMC-AS), polyacrylates, and mixtures thereof, preferably the at least one polymer is preferably selected from the group consisting of HPMC, HPMC-AS, HPC, PVP and PVA, in particular is HPMC or HPMC-AS.

(23) The solid pharmaceutical composition according to anyone of items (18) to (22), wherein in said solid dispersion the weight ratio of compound of formula 1 and the at least one polymer is from about 5:1 to about 1:40, preferably from about 4:1 to about 1:20, more preferably from about 2:1 to about 1:10.

(24) The solid pharmaceutical composition according to anyone of items (1) to (13) and (18) to (23), which comprises a solid dispersion of said compound of formula 1 with a polymer in admixture with at least one further excipient.

(25) The solid pharmaceutical composition according to item (24), wherein by a further excipient in admixture with said solid dispersion granules are formed, preferably by said solid dispersion having been coated on, poured on or otherwise applied onto such further excipient and mixed until granulate is formed.

(26) The solid pharmaceutical composition according to item (24) or (25), wherein a further excipient is selected from the group consisting of water insoluble polymers; inorganic salts and metal silicate materials such as aluminosilicates, preferably aluminometasilicates, more preferably magnesium aluminometasilicates, e.g. Neusilin®; particulate sugars, preferably lactose; cellulose and cellulose derivatives; starch; sugar alcohols; inorganic oxides; preferably sugars such as lactose (monohydrate or anhydrous), cellulose such as microcrystalline cellulose, e.g. Avicel® and silicified microcrystalline cellulose, such as Prosolv®.

(27) The solid pharmaceutical composition according to anyone of the preceding items, wherein the surfactant and/or the polymer is a substance capable of inhibiting precipitation of the compound of formula 1.

(28) The solid pharmaceutical composition according to any one of the preceding items, further comprising one or more other pharmaceutical excipients, wherein said excipients are selected from the group consisting of fillers, disintegrants, binders, lubricants, glidants, film-forming agents and coating materials, sweeteners, flavoring agents, and coloring agents.

(29) The solid pharmaceutical composition according to anyone of the preceding items, wherein all components (a) to (c), and preferably all inactive ingredients originally are solid materials.

(30) The solid pharmaceutical composition according to any one of the preceding items, which has a content of antioxidants below the maximum daily intake limit as foreseen by IIG (status October 2013), preferably is free of the antioxidants butylated hydroxyanisole (BHA) and butylated hydroxytoluene (BHT), more preferably is free of antioxidants.

(31) The solid pharmaceutical composition according to any one of the preceding items, which is in the form of a hard gelatine capsule or a tablet, preferably a film-coated tablet.

(32) The solid pharmaceutical composition according to item (31), wherein one dosage unit of said hard gelatine capsule or said tablet contains the compound of formula 1 in a content of from 10 mg to 480 mg, preferably contains the compound of formula 1 in a content of 40 mg or 160 mg.

(33) A process for the preparation of a solid pharmaceutical composition according to item 1 comprising one or more step(s) of mixing said compound of formula I, the carrier and said surfactant.

(34) The process according to item (33), wherein the one or more step(s) of mixing comprises:

a) providing a solution of the compound of formula 1 in a solvent or mixture of solvents dissolving said compound;

b) mixing a solution of a) with a solid adsorbate carrier, and

c) drying the mixture of b) to thereby yield a solid adsorbate of said the compound of formula 1 being adsorbed on the surface of said solid adsorbate carrier;

d) optionally carrying out further processing steps selected from granulation, compression, tableting, pelletisation, and capsulation, coating, preferably using further excipients where appropriate,

wherein said surfactant is added in any one of steps a) to d).

(35) The process according to item (34), wherein steps a) and b) include dissolving the compound of formula 1 in one or more first solvent(s), preferably halogenated alkanes, in particular dichloromethane or chloroform, then adding the solid adsorbate carrier, and optionally then adding a different second solvent having lower polarity than the first solvent, preferably alkanes, in particular n-hexane, prior to carrying out drying step c).

(36) The process according to item (33), wherein the one or more step(s) of mixing comprises:

a′) providing a solution of the compound of formula 1 in a solvent or mixture of solvents dissolving said compound, and adding a polymer to obtain a solution or dispersion additionally containing the polymer;

b′) optionally mixing the solution or dispersion of a′) with one or more further excipient(s); and

c′) drying the mixture of a′ or b′) to yield a composition comprising a solid dispersion or solid solution of said compound of formula 1 with said polymer;

d) optionally carrying out further processing steps selected from granulation, compression, tableting, pelletisation, and capsulation, coating, preferably using further excipients where appropriate,

wherein said surfactant is added in any one of steps a′) to d).

(37) The process according to item (36), wherein the solvent used for step a′) is selected from the group consisting of ketones and alcohols, preferably is acetone.

(38) The process according to anyone of items (34) to (37), wherein drying step c) is carried out by any one of vacuum drying, by rotary evaporation, freeze drying, fluid bed drying, spray drying, tray drying, microwave drying or other processes resulting in solvent evaporation.

(39) The process according to anyone of items (33) to (38), wherein anyone of the features or conditions set for in items (2)-(4), (7)-(32) is (are) observed.

(40) Solid pharmaceutical composition according to any one of items (1) to (32) for use in the treatment of prostate cancer, in particular in the treatment of male patients with metastatic castration-resistant prostate cancer.

### Definitions

The term “compound of formula 1” as used herein specifically includes Enzalutamide or ARN-509 as well as very closely related compounds expected to have same properties, including activity as androgen receptor antagonist. The active compound occasionally in the present specification may be altogether also named “API” or “API compound”.

Preferably, the compound of formula 1 as meant in all aspects, embodiments and descriptions disclosed herein is Enzalutamide denoted by the following formula (hence in formula 1 X═C and Y₁═Y₂═CH₃):

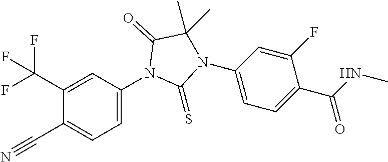 [SMILES: CNC(=O)c1ccc(N2C(=S)N(c3ccc(C#N)c(C(F)(F)F)c3)C(=O)C2(C)C)cc1F]

or is ARN-509, denoted by the following formula (hence in formula 1 X═N and Y₁ and Y₂ are interconnected to form a cyclobutane ring compound):

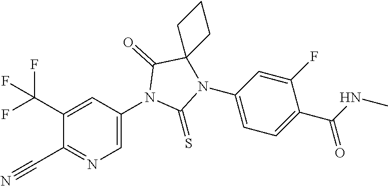 [SMILES: CNC(=O)c1ccc(N2C(=S)N(c3cnc(C#N)c(C(F)(F)F)c3)C(=O)C23CCC3)cc1F]

In the context of the present invention, the term “amorphous compound of formula 1”, “amorphous Enzalutamide” or “amorphous ARN-509” indicates that the respective compound is present in the composition or in parts thereof (i.e. the pharmaceutical composition, the solid dispersion, or the adsorbate) in mainly amorphous state, preferably substantially amorphous state. “Mainly” amorphous denotes “more than 50%”, “substantially” amorphous denotes that at least 90%, preferably 95% or 97%, more preferably all of the respective compound is amorphous. In other words, “amorphous” means minor amounts and preferably no substantial amounts, more preferably no noticeable amounts, of crystalline portions of the respective compound, as e.g. measurable upon X-ray powder diffraction (XRPD) analysis. In order to assess whether an entire, final API-containing composition according to the present invention comprises only or substantially only amorphous API, the XRPD pattern of the given composition may be compared with the XRPD pattern of a placebo-composition, i.e. the composition without the active API compound; if then both the API-containing composition and placebo-composition correspond to each other in XRPD, the API should be present in amorphous form only. Specifically, XRD measurements are carried out firstly with the crystalline counterpart form as a reference, secondly with the other relevant component alone (adsorbate substrate or polymer used for solid dispersion) also as a reference, and thirdly with the sample in question, and then the measurement results are compared. If the sample measurement and XRPD results correspond to the second reference, without the presence of “crystalline” peaks of the first reference, then amorphous form is confirmed. Amorphous ratio is determined depending on the degree/magnitude of “crystalline” peaks in the sample in question.

The term “surfactant” used herein is, as generally understood by persons skilled in the art, a substance which per se can lower the surface tension (or interfacial tension) between two liquids or between a liquid and a solid. Preferably, the term “surfactant” used herein means a substance capable of acting as wetting agent, as emulsifier, as detergent, and as dispersant, more preferably a substance capable of acting as wetting agent. The general function of a substance being a surfactant may be typically known in advance by a skilled person. More specifically, the aforementioned capacity of the surfactant to be used may be tested by simple measurements of whether the dissolution of the compound of formula 1 in a given composition or formulation can be enhanced compared with the same composition or formulation but without the surfactant under same defined conditions such as dissolution medium, temperature and stirring conditions, for example the herein preferred dissolution test in fasted state simulated intestinal fluid (FaSSIF) pH 6.5 medium at 45 minutes and at 100 rpm in USP Apparatus 2 (paddle method). Suitable, preferred and most preferred surfactants to be used in the present invention are further described herein elsewhere.

A “carrier” within the meaning of the present invention may also refer to herein as “particles of a carrier” or “carrier particles”. In specific embodiments as further described elsewhere, a carrier for an “adsorbate” is the solid adsorbate support material, whereas a carrier for the solid solution or solid dispersion is a suitable polymer. As self-evidently understood from the definition of the three enumerated ingredients (a), (b) and (c), inactive ingredients (b) and (c) each are respectively additive to the active ingredient (a). That is, the carrier is used in the present invention further to the surfactant. The respective substances are different to accomplish their respective function. Further conventional excipients may be mixed therewith, such as filler, disintegrant, binder, lubricant, glidant, etc., as also further described elsewhere.

The expression “adsorbate”, as used herein, specifies that the the compound of formula 1, notably Enzalutamide or ARN-509 is—preferably evenly and preferably homogeneously —distributed on inner and/or outer surfaces of the particulate substrate (sometimes also named adsorbate substrate). The presence and the distribution of the API on the surface of a substrate can be analyzed for instance by Raman imaging, XPS or ESCA. The API is preferably adsorbed to the substrate in a layer on its (outer and optionally also inner) surface; layer thickness may range from a monolayer or layer on a molecular level, extending to larger thicknesses in the nm and μm range, up to e.g. about 50 μm. This may also depend on the type of substrate. Further, inactive excipients such as surfactants and polymers may be included within the layer of API adsorbed on the substrate, which may lead to a correspondingly increased layer thickness. In a preferred embodiment of the adsorbate, the API is deposited on the inner and/or outer surface of a suitable substrate, wherein the API is in its free form, and/or no API particles or API precipitates are formed on the substrate. When preparing the adsorbate, a solution where the compound of formula 1, notably Enzalutamide or ARN-509 is dissolved, preferably completely dissolved in a selected solvent or mixture of solvents, is applied onto the solid support, and subsequently the solvent or mixture of solvents is removed, typically by evaporating. A possibility of applying the compound of formula 1 onto the solid support (the adsorbate carrier) includes dissolving the compound of formula 1 in one or more first solvent(s), then adding the solid adsorbate carrier, and then performing solvent evaporation/drying. As a further, preferred manner prior to solvent evaporation/drying, a different second solvent having lower polarity than the first solvent(s) is (are) added. In the last-mentioned preferred embodiment, the change into a solvent system of reduced polarity effectively forces the compound of formula 1 to adhere to the surface of the solid support. Even more preferably, the addition of the second solvent is made slowly for promoting a controlled adsorbance process and thereby to achieve a high proportion of the compound of formula 1 being in amorphous form. A controlled adsorbance process also favours the compound of formula 1 to be stabilized in the adsorbate form.

Within the meaning of the present invention, the term “solid dispersion” (or “solid solution”), denotes a state of the compound of formula 1, notably Enzalutamide or ARN-509, where most of it, preferably 90%, 95% or all of the compound present in the solid dispersion is molecularly dispersed in a solid polymer, which acts as a carrier, typically forming a homogeneous one-phase system with a polymer matrix. Preferably the active compound is reduced to its molecular size in the solid dispersion or solid solution, or at most nm-sized API particles. In a preferred embodiment of the present invention, the solid dispersion is a solid solution.

In order to characterize the physical nature of solid dispersions, techniques such as thermal analysis (such as cooling curve, thaw melt, thermo microscopy and DTA methods), x-ray diffraction, microscopic methods, spectroscopic methods, dissolution rate, and thermodynamic methods can be used. It is also possible to use two (or even more) of the above listed methods in order to obtain a complete picture of the solid dispersion system, if need be.

In case of the preferred embodiment of the present invention where the solid dispersion/solid solution is carried on a further excipient or is mixed with other constituents/components, the above definition relates to the true solid dispersion/solid solution part; other constituents/components or other excipients optionally present in the whole pharmaceutical composition may be disregarded for the status characterization of the solid dispersion/solid solution.

The terms “about” or “substantially” in the context of the present invention denote an interval of accuracy that the person skilled in the art will understand to still ensure the technical effect of the feature in question. The term “about” typically indicates deviation from the indicated numerical value of ±10%, and preferably ±5%.

### DETAILED DESCRIPTION OF THE INVENTION

The present invention is now described in more detail by preferred embodiments and examples, which are however presented for illustrative purpose only and shall not be understood as limiting the scope of the present invention in any way.

The present invention overcomes shortcomings of the prior art formulations of the marketed liquid Enzalutamide composition filled in capsules (Xtandi®), which requires high amounts of Labrasol®, or of crystalline ARN-509-filled capsule composition known from WO 2013/184681 A1 which is associated with poor API solubility, by providing entirely solid versions of a pharmaceutical composition from which the API compound, notably Enzalutamide or ARN-509, quickly dissolves or is released, thereby ensuring high bioavailability and effectiveness, especially in bio-relevant media as tested in simulated gastric or intestinal fluid. By enabling such solid dosage forms but without sacrificing solubility performance, the present invention offers protection of patient or other persons in contact with the dosage form against leakage on breaking or other physical contact with active ingredient. Further, it is possible that the pharmaceutical compositions of the compound of formula 1, notably of Enzalutamide or ARN-509, can be made small in physical volume if desired, in order to be easily swallowable by patients and come in a small number of units per daily recommended dose, preferably in single dosage unit(s), thereby enhancing patient compliance. Even more surprisingly, these advantages can be accomplished at low content ratio of the surfactant relative to the compound of formula 1, thereby remarkably lowering bio-burden e.g. compared to the marketed product Xtandi®. Further, it is even possible that the advantages of the pharmaceutical composition of the invention can be achieved without or at lower contents of antioxidants and/or other ingredients that may significantly elevate bio-burden to patients undergoing drug therapy. Hence, the solid pharmaceutical composition of the present invention has remarkably improved overall pharmaceutical attributes.

Moreover, despite solubility and stability challenges of the API compounds involved, it was surprisingly found that the pharmaceutical composition of the present invention can be formulated at affordable costs and in a robust manner, i.e. can be processed with common pharmaceutical technologies such as mixing, granulation, tableting, pelletisation, capsulation, coating and similar.

It is particularly beneficial that advantages of the present invention can be achieved at a relatively low ratio of the surfactant relative to the compound of formula 1, specifically being not higher than 10:1, preferably not higher than 5:1, more preferably not higher than 2:1, for example in beneficial ranges of 5:1 to 1:10, preferably 3:1 to 1:5, more preferably 2:1 to 1:2. By this limited ratio, and depending on a desired dosage of the compound of formula 1, the total amount of surfactant in the whole composition can be kept relatively low, yet can lie in a beneficial range of at least 0.5 wt. % while observing the aforementioned ratio of the API compound.

Particularly suitable surfactants as component (c) can be selected from the group consisting of anionic surfactants, preferably sodium lauryl sulphate; polyethylene glycols (PEGs), preferably those PEGs having molecular weight in the range of about 2000 to 10000, more preferably PEG 3350, PEG 4000, PEG 6000, PEG 8000; Polysorbates, preferably Tween 20, Tween 80 or Span 80; fatty acid esters, preferably propylene glycol caprylates such as Capmul PG-8, Capryol 90; esters of glycerol and fatty acids, preferably glycerol oleates and caprylates (Capmul MCM); esters of polyethylene glycol and fatty acids, such as Labrasol and Solutol; castor oil ethoxylate (glycerol polyethylene glycol ricinoleate) such as Cremophor EL and Cremophor RH 40. More preferably the surfactant is selected from the group consisting of sodium lauryl sulphate; PEG 3350, PEG 4000, PEG 6000 or, PEG 8000 and preferably PEG 6000; Tween 20 or Tween 80; and esters of polyethylene glycol and fatty acids, most preferably sodium lauryl sulphate and PEG 6000 and in particular sodium lauryl sulphate.

Furthermore, use of a surfactant which per se is a solid substance, and limiting an amount of surfactant even if per se liquid, provides an advantage by contributing to produce a entirely dry and solid pharmaceutical composition. As suitable per se solid surfactants sodium lauryl sulphate, dry type fatty esters of the surfactant substances mentioned above, etc.

In preferred embodiments of the solid combination of the API compound, the carrier and the surfactant according to the present invention, the compound of formula 1 and the carrier are in association with each other, without separation therebetween. By this means a proportion of amorphous phase of the API compound can be increased or even can be made and kept in mainly and preferably substantially or even totally in amorphous phase, which not only favours dissolution of the API, but in addition can assist in stabilization of the compound of formula 1. Furthermore in favour of dissolution properties, a proper and intimate association, especially by means of the adsorbate and solid dispersion embodiments further described below, can preferably effect that compound 1 is present in the composition not in the form of particles (at least coarse particles), not in the form of precipitate, and/or not in crystalline form (at least substantially).

It has been found that a particularly effective and beneficial association between the compound of formula 1 and a carrier can be realized by a combination of components (a) and (b) in the form of a solid adsorbate in which the active compound is adsorbed on the surface of a carrier. More surprisingly, it has been found that dissolution of the compound of formula 1 is significantly enhanced by a combination of the adsorbate with the surfactant, in particular in comparison with a much inferior dissolution rate obtained when the compound of formula 1 is combined with the same surfactant however without being present on the adsorbate carrier.

Therefore, a carrier for the adsorbate (i) has an outer and/or inner surface onto which the compound of formula 1 can be adsorbed. When initially porous as the preferred choice, the pores of the adsorbate carrier are, at least partially, filled by the compound of formula 1 by the adsorption process. Furthermore, the carrier in the adsorbate used according to the present invention may not, at least not essentially, change its morphology during and after the adsorption of the compound of formula 1, i.e. the physical shape and outer structure of the adsorbate corresponds to, at least essentially corresponds to, the physical shape and outer structure of the substrate alone. This criterion is an indication that a thin layer, even down to a monolayer but also up to higher layer thickness, is formed on the—outer and/or inner—surfaces of the substrate, which favors compound dissolution. It may be further indicative of a minimization or absence of more difficult to dissolve coarse particles, precipitates and/or crystals of the API compound.

A desirable porosity can be determined according to DIN EN 623-2, wherein the porosity is preferably at least 20%, 30%, 40%, 50% or 60%. Also preferred, the porosity is in the range of between 10-70%, further preferred between 20-70%, even further preferred between 30-70% or between 40-70%. The term “porosity” as used herein refers to the open pore porosity, which can be determined using the aforementioned method. The open pores of the substrate will typically be accessible to the solvent containing the API during the process for preparation of the adsorbates.

It is further preferred that the substrate has a high BET-surface area. A person skilled in the art knows what BET-surface area is “high”, respectively based on the BET-surface areas the respective substrate can have. For instance, the BET-surface area is at least 10 m²/g, s preferably at least 50 m²/g, more preferably at least 250 m²/g. The determination of the BET-surface area of the substrate can be carried out according to known methods, for example as described in the article: J. Am. Chem. Soc. 60, 309 (1938). Additionally, the substrates with the defined BET-surfaces can have a porosity as defined above. A decrease of the BET-surface area in the comparison before and after the API adsorption process may be an indication that the surface layer of the substrate could be effectively loaded with the API, and consequently its porosity and specific surface area decreases correspondingly. The obtained adsorbate can for instance be analyzed by SEM (magnification e.g. 100 times to 10000 times) or Raman imaging.

The material for the carrier for the adsorbate can be suitably selected from particulate and/or porous substrate inorganic oxides and particulate and/or porous substrate water insoluble polymers. Substances for the particulate inorganic oxides may be selected from the group consisting of SiO₂, TiO₂, ZnO₂, ZnO, Al₂O₃, CaCO₃, Ca₂(PO₄)₂ and zeolite, preferably the inorganic oxide is particulate SiO₂, more preferably colloidal or fumed silicon dioxide or porous silica. Commercially available examples for suitable carriers are Aerosil® 90, 130, 150, 200 or 380 or Aerosil® OX 50, EG 50 or TT 600 (Evonik Degussa GmbH, Germany), or Syloid series such as Syloid 244 or Syloid AL-1 (Grace Davison, USA), HDK pyrogenic silica series such as HDK N20 (Wacker Chemie AG, Germany), Porasil and Lichrosorp can be used. Preferably Aerosil® 200 or Syloid 244 can be used, more preferably Syloid AL-1 can be used. As a carrier of the water insoluble polymer type, silicified microcrystalline cellulose may be mentioned, e.g. the material obtainable from JRS Pharma, sold under the trade name PROSOLV® SMCC.

It is possible to adjust a content of API compound in the adsorbate in a way beneficial for both dissolution and/or stabilization. For example a suitable amount of the compound of formula 1 in the adsorbate lies in a range of about 2 to about 35 wt.-%, preferably in the range of about 3 to about 30 wt.-%, more preferably in the range of about 5 to about 25 wt.-%, and even more preferably in the range of about 10 to about 20 wt.-%, respectively in % by weight relative to the whole adsorbate.

According to further, preferred embodiment of the present invention, to be used alone or in combination with other embodiments described herein, the solid pharmaceutical composition comprises the compound of formula I, in particular Enzalutamide or ARN-509 specifically, in the form of a solid dispersion with a polymer. The polymer for said solid dispersion is suitably selected from a hydrophilic polymer, preferably a water-soluble polymer. A preferred polymer is one which allows the compound of formula 1 to be presented in mainly, preferably essentially and most preferably entirely in amorphous form in the solid pharmaceutical composition and beneficially kept for long time in such form. Accordingly the solid dispersion can be formed with at least one polymer selected from the group consisting of hydroxyethylcellulose (HEC), hydroxypropyl cellulose (HPC), hydroxypropyl methyl cellulose (HPMC), polyvinyl pyrrolidone (PVP), polyvinyl alcohol (PVA), polyacrylic acid (PAA), poly(ethylene glycol) (PEG), poly(ethylene oxide) (PEO), copovidone, hypromellose acetate succinate (HPMC-AS), polyacrylates, gum arabic, xanthan gum, tragacanth, acacia, carageenan, guar gum, locust bean gum, pectin, alginates, and mixtures thereof. Preferably the at least one polymer is selected from the group consisting of HPMC, HPC, PVP and PVA, in particular is HPMC or HPMC-AS.

When said polymer is selected from appropriate hydrophilic cellulose derivatives and PVA, it may serve not only as a suitable matrix polymer for the solid dispersion, but at the same time may additionally act as a wettability enhancer.

In particularly preferred embodiments, the polymer is chosen by which the compound of formula 1 is contained in the form of a solid solution, and/or that the solid dispersion of the polymer and the compound of formula 1 is substantially homogeneous. The potential of the water-soluble polymer to co-dissolve the compound of formula 1 can be enhanced with increased polymer-compound interaction and/or embedding of the compound in the polymer.

In the solid dispersion of the compound of formula 1 mixed with the polymer, the weight ratio of compound of formula 1 and the at least one polymer suitably lies in a range from about 5:1 to about 1:40, preferably from about 4:1 to about 1:20, more preferably from about 2:1 to about 1:10.

In order to obtain the solid dispersion, preferably a solid solution, a desirable minimum proportion of the compound of formula 1 is dissolved in a solvent or mixture of solvents suitable for dissolving it, at least at one time point during preparation of said solid dispersion. After such liquid solution is made and a polymer is added, solvent(s) is (are) removed and the mixture is dried. Accordingly a solid dispersion or solid solution within the meaning of the present invention can be generated. A “desirable minimum proportion of the compound of formula 1” means that at least 80%, preferably at least 90%, and more preferably at least 95% of originally used compound should preferably be dissolved in a suitable solvent. Further the polymer should be dispersed in the solvent(s). Preferably, all of the used compound and all of the polymer are entirely dispersed when preparing the solid dispersion.

When in a preferred embodiment the solid dispersion is admixed with a further excipient, which preferably is a further particulate substance, granules can be formed in which the solid dispersion or solid solution is present, at least in part, on such particulate substance, thereby providing a useful product or intermediate product. A suitable process to obtain such granulate may include dissolution of the API compound in a solvent, addition of polymer in a an appropriate solvent, contacting an obtained mixture thereof with the further excipient such as one or more filler, granulating the obtained mixture, optionally additionally admixing with further excipients such as disintegrants, and finally removing solvent by solvent evaporation and optionally drying.

An excipient particularly suitable for being admixed with the solid dispersion may be selected from the group consisting of water insoluble polymers; inorganic salts and metal silicate materials such as magnesium aluminosilicates, e.g. Neusilin®; sugars and sugar alcohols. Water insoluble polymer may be selected from the group consisting of cross-linked polyvinyl pyrrolidinone, cross-linked cellulose acetate phthalate, cross-linked hydroxypropyl methyl cellulose acetate succinate, microcrystalline cellulose, polyethylene/polyvinyl alcohol copolymer, polyethylene/polyvinyl pyrrolidinone copolymer, cross-linked carboxymethyl cellulose, sodium starch glycolate, and cross-linked styrene divinyl benzene. Preferably the water insoluble polymer is starch and starch derivatives, water insoluble cellulose derivatives and microcrystalline cellulose (e.g. Avicel®); and the preferable sugar is lactose (monohydrate or anhydrous).

Beneficially, when appropriately selected such a further excipient can also act to additionally increase wettability of the whole composition, for example when using appropriate particulate sugars and sugar alcohols such as lactose and/or or appropriate particulate inorganic substances, for example Neusilin®.

As with other embodiments, also the solid pharmaceutical composition using the above described solid dispersion contains the compound of formula 1 in mainly amorphous form, preferably substantially amorphous form, and does not contain substantial amounts, preferably does not contain noticeable or measurable amounts, of crystalline portions of the compound of formula 1, as e.g. measurable upon X-ray powder diffraction (XRPD) measurement. As another suitable method to determine whether the compound of formula 1 is amorphous in the solid dispersion, DSC may be used where a lack of a significant melting peak may be indicative of no a only insignificant crystalline proportion (usually <2%) of the compound.

Furthermore, in favour of dissolution properties compound 1 is present in the solid dispersion not in the form of particles, and/or not in the form of precipitate. The presence (or absence) of particles or precipitate of the compound of formula 1 can be assessed by any suitable method that is known to a person skilled in the art, for instance by Raman imaging, by electron microscopic observation (such as scanning electron microscopy, SEM) or the like.

Based on BCS classification of Enzalutamide, dissolution is key factor for Enzalutamide bioavailability. On the other hand, based on physiologically based pharmacokinetic (PBPK) model it can be concluded that bioavailability of Enzalutamide is not significantly affected by dissolution from dosage form at short times. The PBPK model built on published in vivo data (“NDA 203415 Review Xtandi™-Enzalutamide, Clinical Pharmacology and Biopharmaceutics Review (s), FDA”), and solubility in FASSIF medium with GastroPlus™ 8.0 software (Simulations Pllus, Inc.). With the use of the developed model, time profile of Enzalutamide plasma concentration after Enzalutamide single dose intake was calculated for Xtandi and examples 5, 6 and 13 (see FIG. 6).

The model confirms in vivo precipitation of Xtandi and gradual in vivo dissolution of samples from Examples 5, 6 and 13 The difference in rate and amount of absorbed Enzalutamide from Xtandi and other examples is not therapeutically significant, with Cmax and AUC ratios above 80%. Based on simulations with the model, the dissolution threshold to obtain bioavailability comparable to that of Xtandi was set to NLT 35% of Enzalutamide dose dissolved in 500 ml of FaSSIF pH 6.5 medium in 45 minutes.

It became possible by the present invention that solubility of the compound of formula 1 was surprisingly improved in bio-relevant media as tested in simulated gastric or intestinal fluid. The solid pharmaceutical composition according to the present invention thus can achieve a desirable minimum dissolution ratio of the compound of formula 1 of not being less than (NLT) 35%, more preferably NLT 40% or even higher thresholds, when the pharmaceutical composition is subjected to a dissolution test in fasted state simulated intestinal fluid (FaSSIF) pH 6.5 medium at 45 minutes and at 100 rpm in USP Apparatus 2 (paddle method).

Preferably, the solid pharmaceutical composition according to present invention comprises a substance capable of inhibiting precipitation of the compound of formula 1. More preferably, the surfactant and/or the polymer is chosen such that it also acts as such a substance capable of inhibiting precipitation of the compound of formula 1. Whether a substance has such capacity can be determined by a simple reference test when choosing such a substance in advance of incorporating it into the final composition. For this purpose, a saturated solution of the desired compound of formula 1 (e.g. 12 mg of Enzalutamide) is made by completely dissolving it in a suitable solvent of limited volume (e.g. 0.27 ml of Tween 80) together with the suitable amount of chosen test substance. This solution is then transferred to a higher volume of medium which allows for adequate discrimination between different test substances at physiological pH values (such as pH 6.8 phosphate buffer). The quantity of the medium is chosen to reflect the dissolution of full dose compound of formula 1 in physiological volume of about 250 ml. E.g., 12 mg of Enzalutamide is first dissolved in 0.27 ml of Tween 80 to form a saturated solution. To this solution 0.15 mg of test substance (hydroxypropyl methyl cellulose) is added. The solution is then transferred to 15 ml of pH 6.8 phosphate buffer, corresponding to dissolution of about 200 mg of Enzalutamide in 250 ml of medium. This transfer represents a zero point and from this point onwards the concentrations of still dissolved compound are measured, repeating the dissolved concentration measurement at time points from 10 to 360 minutes. Time dependent decrease of concentration in pH 6.8 phosphate buffer is a measure of precipitation of the compound. Best precipitation inhibiting capability is identified for those test substances or solvents that deliver highest concentrations of the compound of formula 1 in test medium at individual time points.

By additionally observing this feature, it is possible that the compound of formula 1, notably Enzalutamide or ARN-509, once dissolved remains dissolved without or with reduced precipitation. A substance capable of inhibiting precipitation of the compound of formula 1 can be chosen from appropriate polymers, suitably hydrophilic and water-soluble polymers. Further preferred, precipitation inhibition may concurrently be accomplished if appropriate surfactants and/or precipitation inhibiting polymers are chosen to be present in the composition, such as HPMC, HPC, PVA, PVP or PEG. Particularly beneficial precipitation inhibition has been found by a combination of the API compound with surfactant and hydrophilic water soluble polymer, for example HPMC, leading to a remarkably enhanced solution stability compared with the respective surfactant alone.

The stability of the compositions of the present invention is particularly ensured with a solid formulation in which no ingredient remains in liquid form. This significantly reduces the contact between particles of different ingredients, leading to smaller probability of reactions that induce degradation of the active ingredient. Therefore, preferably all components (a) to (c), and more preferably all inactive ingredients originally are solid materials.

The solid pharmaceutical composition according to the present invention may further comprise one or more other pharmaceutical excipients. Useful excipients other than those, or further amounts of the same already described substances exerting the beneficial functions above but additionally displaying one or more further functions, may be selected from the group consisting of typical fillers, disintegrants, binders, lubricants, glidants, film-forming agents and coating materials, sweeteners, flavoring agents, plasticizers, and coloring agents such pigments. Other excipients known in the field of pharmaceutical compositions may also be used.

Fillers, if used (optionally in addition to the already described functions), may be selected from the group consisting of different grades of starches, such as maize starch, potato starch, rice starch, wheat starch, pregelatinized starch, fully pregelatinized starch; cellulose derivatives, such as microcrystalline cellulose or silicified microcrystalline cellulose; sugar alcohols such as mannitol, erythritol, sorbitol, xylitol; monosaccharides like glucose; oligosaccharides like sucrose and lactose such as lactose monohydrate, lactose anhydrous, spray dried lactose or anhydrous lactose; calcium salts, such as calcium hydrogenphosphate; particularly preferably the fillers are selected from the group consisting of, microcrystalline cellulose, silicified microcrystalline cellulose, lactose monohydrate, spray dried lactose, and anhydrous lactose.

Disintegrants, if used (optionally in addition to the already described functions), may be selected from the group consisting of carmellose calcium, carboxymethylstarch sodium, croscarmellose sodium (cellulose carboxymethylether sodium salt, crosslinked), starch, modified starch such as pregelatinized starch, starch derivatives such as sodium starch glycolate, crosslinked polyvinylpyrrolidone (crospovidone), and low-substituted hydroxypropylcellulose, and disintegrating aids such as magnesium alumino-metasilicate and ion exchange resins like polacrilin potassium; particularly preferably the disintegrants are selected from the group consisting of sodium starch glycolate, croscarmellose sodium and crospovidone.

Lubricants, if used, may be selected from the group consisting of stearic acid, talc, glyceryl behenate, sodium stearyl fumarate and magnesium stearate; particularly preferably the lubricant are magnesium stearate and sodium stearyl fumarate.

Binders, if used (optionally in addition to the already described functions), may be selected from the group consisting of polyvinyl pyrrolidone (Povidone), polyvinyl alcohol, copolymers of vinylpyrrolidone with other vinylderivatives (Copovidone), hydroxypropyl methylcellulose, methylcellulose, hydroxypropylcellulose, powdered acacia, gelatin, guar gum, carbomer such as carbopol, polymethacrylates and pregelatinized starch.

Diluents, if used, may correspond to the fillers listed above.

Glidants, if used, may be selected from the group consisting of colloidal silica, hydrophobic colloidal silica and magnesium trisilicate, such as talc; particularly preferably the glidants are selected from the group consisting of colloidal silica and hydrophobic colloidal silica.

Suitable sweeteners may be selected from the group consisting of aspartame, saccharin sodium, dipotassium glycyrrhizinate, aspartame, stevia, thaumatin, and the like.

Preferably, if used (optionally in addition to the already described functions), the further used excipients are microcrystalline cellulose, silicified microcrystalline cellulose, anhydrous lactose, lactose monohydrate, spray dried lactose, croscarmellose sodium, sodium starch glycolate, low substituted hydroxypropylcellulose, crospovidone, magnesium stearate, and sodium stearyl fumarate.

Suitable film-forming agents and coating materials, if used, e.g. for the preparation of film coatings on API-containing tablets, may include, but are not limited to hydroxypropyl methylcellulose (hypromellose, HPMC), hydroxypropyl cellulose, polyvinylalcohol, methylcellulose, ethylcellulose, hydroxypropylmethyl cellulose phthalate, hydroxypropylmethyl cellulose acetate succinate, shellac, liquid glucose, hydroxyethyl cellulose, polyvinylpyrrolidone, copolymers of vinylpyrrolidone and vinylacetate such as Kollidon® VA64 BASF, copolymers of acrylic and/or methacrylic acid esters with trimethylammoniummethylacrylate, copolymers of dimethylaminomethacrylic acid and neutral methacrylic acid esters, polymers of methacrylic acid or methacrylic acid esters, copolymers of acrylic acid ethylester and methacrylic acid methyl ester, and copolymers of acrylic acid and acrylic acid methylester.

Plasticizers, if used, may include, but are not limited to polyethylene glycol, diethyl phthalate and glycerol. Preference is given to polyethylene glycol.

Suitable coloring agents, if used, may include, but are not limited to pigments, inorganic pigments, FD&C Red No. 3, FD&C Red No. 20, FD&C Yellow No. 6, FD&C Blue No. 2, D&C Green No. 5, D&C Orange No. 5, D&C Red No. 8, caramel, ferric oxide red, ferric oxide yellow and titanium dioxide.

Specifically, a particularly beneficial feature that can be achieved by the solid pharmaceutical composition according to the present invention is compactness in size. Accordingly, it is made possible according to the present invention that preferably a full recommended daily dose of Enzalutamide (160 mg) can be formulated in a single dosage form, or in a few dosage units in order to meet the desired or recommended daily dose, e.g. 4-fold a 40 mg dosage unit per day. In order to realize this, according to a further preferred embodiment excipients with hybrid or multitude of functions have been found to be successfully selected, achieving as much as possible of the aforementioned useful problem-solving and excipient functions.

In Xtandi, the patient digests four soft gelatine capsules, each with a volume of about 1.3 cm³, for a full daily dose of Enzalutamide (160 mg). In the compositions of the present invention it is possible to reduce the volume of individual dosage units (40 mg of Enzalutamide) to 0.6 cm³ or lower, even down to as low as 0.17 cm³ or below, while still conforming to the desirable dissolution criterion. The latter value represents an over 7-fold improvement over the existing marketed formulation and makes it possible for the highest recommended daily dose of Enzalutamide (160 mg) to be formulated as a single tablet with weight as low as 680 mg.

Thus, the solid pharmaceutical composition according to the present invention, as advantage in comparison with the prior art possess a high drug load. Preferably the amount of the compound of formula 1 in the entire composition is greater than 5%, more preferably greater than 10%, even more preferably greater than 15%.

Further preferably, thanks to acceptable to even good stability performances achievable by the solid pharmaceutical composition according to the present invention, it is possible that antioxidants, which had been required in the marketed Xtandi product, is used in relatively reduced amounts, or that the composition is even free of antioxidants, preferably free of artificial antioxidants and in particular free of the antioxidants butylated hydroxyanisole (BHA) and butylated hydroxytoluene (BHT).

As suitable dosage forms, the solid pharmaceutical composition according to the present invention is in the form of a capsule or a tablet, preferably a capsule or a film-coated tablet. For example, a capsule such as a gelatine capsule may be filed with granulate formed with the solid dispersion or the adsorbate described above, or a tablet is compressed involving the uses of such granulate and optionally further film-coated, respectively and optionally with conventional excipients useful for such technologies.

The solid pharmaceutical composition according to the present invention is particularly useful in medical treatments, specifically in the treatment of prostate cancer and in particular in the treatment of male patients with metastatic castration-resistant prostate cancer.

Moreover it was found according to another aspect of the present invention that solid compositions or formulations of the compound of formula 1, notably of Enzalutamide and ARN-509, can be prepared in simple and robust manner, allowing to use common pharmaceutical technologies at relatively low costs.

According to this other aspect, the preparation process may simply comprise one of more step(s) of mixing said compound of formula I, the carrier and said surfactant.

Particularly, the process for the preparation of the solid preparation of a compound of formula 1, including Enzalutamide and ARN-509, can comprise the steps of:

a) providing a solution of the compound of formula 1 in a solvent or mixture of solvents dissolving said compound, preferably using halogenated alkanes, in particular dichloromethane or chloroform;

b) mixing a solution of a) with a solid adsorbate carrier, preferably including adding a different second solvent having lower polarity than the solvent used in step a), more preferably adding an alkane;

c) drying the mixture of b) to thereby yield a solid adsorbate of said the compound of formula 1 being adsorbed on the surface of said solid adsorbate carrier; and

d) optionally carrying out further processing steps selected from granulation, compression, tableting, pelletisation, and capsulation, coating, preferably using further excipients where appropriate,

wherein said surfactant is added in any one of steps a) to d);

or

a′) providing a solution of the compound of formula 1 in a solvent or mixture of solvents dissolving said compound, and adding a polymer to obtain a solution or dispersion additionally containing the polymer as a carrier, wherein preferably the solvent used for step

a′) is selected from the group consisting of ketones and alcohols, more preferably is acetone;

b′) optionally mixing the solution or dispersion of a′) with one or more further excipients,

c) drying the mixture of a′) or b′) to yield a composition comprising a solid dispersion or solid solution of said compound of formula 1 with said polymer; and

d) optionally carrying out further processing steps selected from granulation, compression, tableting, pelletisation, and capsulation, coating, preferably using further excipients where appropriate,

wherein said surfactant is added in any one of steps a′) to d).

The drying step c), as already described previously, serves for evaporating the solvent(s) and may be carried out by any one of vacuum drying, by rotary evaporation (preferably under vacuum), freeze drying (lyophilisation), fluid bed drying, spray drying, tray drying, microwave drying or other processes resulting in solvent evaporation, respectively preferably involving evaporation of the solvent in the respective drying step at a relatively slow speed.

A solvent for any of steps a), a′), b) or b′) can be suitably selected according to the circumstances. Preferably for the steps of mixing under a) and b) include completely dissolving the compound of formula 1 in one or more first solvent(s), preferably halogenated alkanes, in particular dichloromethane or chloroform, then (optionally but preferably) adding the solid adsorbate carrier, and (optionally) then adding a different second solvent having lower polarity than the first solvent, preferably alkanes, in particular n-hexane. For step a′) when preparing the solid dispersion/solid solution, the solvent can be suitably selected from the group consisting of ketones and alcohols, preferably is acetone.

Moreover, at respective appropriate steps, useful substances and/or excipients as described above in detail may be further added.

The oral solid dosage form of the present invention is preferably a compressed or a non-compressed dosage form. Preferably, the oral solid dosage form of the present invention is a granulate, a capsule, for example a capsule filled with granules, a sachet, a pellet, a dragee, a lozenge, a troche, a pastille, or a tablet, such as an uncoated tablet, a coated tablet, an effervescent tablet, a soluble tablet, a dispersible tablet or an extrudate. More preferred dosage forms are capsules filled with API-containing granulate, or compressed dosage forms such as a tablet. Tablets can be prepared by compressing uniform volumes of particles or particle aggregates or granulates, preferably produced by granulation methods. Most preferably, the pharmaceutical composition is an immediate release tablet. Also most preferably, the compound of formula 1 and notably of Enzalutamide and ARN-509 is present in the prepared pharmaceutical composition in pure amorphous form.

### BRIEF DESCRIPTION OF THE DRAWINGS

FIG. 1 shows a comparison of Enzalutamide dissolution from Xtandi (Reference Example 1) and compositions from Examples 5, 6 and 13;

FIG. 2 shows a comparison of Enzalutamide dissolution from Reference Examples 3-6;

FIG. 3 shows a comparison of Enzalutamide dissolution from Examples 3 and 4 and Reference Example 8

FIG. 4 shows a comparison of Enzalutamide dissolution from Example 13 and Reference Example 9;

FIG. 5A-5C show XRD diffractograms to demonstrate entirely amorphous Enzalutamide in adsorbate (Example 1a; FIG. 5A), solid dispersion of Enzalutamide (Reference Example 9; FIG. 5B), and ARN-509 in adsorbate (Example 10; FIG. 5C);

FIG. 6 shows simulated time profiles of Enzalutamide plasma concentrations after Enzalutamide single dose intake for Xtandi (reference example 1) and Examples 5, 6 and 13

### EXAMPLES

After description of dissolution testing methods and stability testing methods, subsequently experiments, Examples and Reference Examples will be described.

Drug Release Testing

To assess bio-availability of prepared examples, we measured dissolution rate of API in FaSSIF (fasted state simulated intestinal fluid) with pH 6.5. This medium contains bile salts, which mimics gastrointestinal conditions. Thus, in-vitro dissolution testing in FaSSIF is applicable for prediction of bioavailability. Dissolution performance of prepared samples were compared to Xtandi or/and Enzalutamide API. A threshold has been set for acceptable dissolution, which ensures required level of bioavailability, as NLT 35% of the dose dissolved in FaSSIF pH 6.5 at 45 minutes. Apparatus 2 (paddle method); 100 rpm and 500 ml of dissolution media has been used.

Stability Testing

Enzalutamide degradation products were followed by high performance liquid chromatography using the following chromatographic method:

Formulations were dissolved in a mixture of 50 w/w % acetonitrile in water to achieve a concentration of about 0.4 mg/ml of Enzalutamide. The sample solution was injected into an HPLC system with a BEH Shield RP18 column (1.7 micrometer particles) using binary gradient elution. Mobile phase A consisted of 0.05% trifluoroacetic acid in water and mobile phase B consisted of 0.05% trifluoroacetic acid in acetonitrile. Gradient elution was performed according to the following program: mobile phase A (%)/time (min): 80%/0 min; 20%/5 min; 80%/5.5 min. The detector was set to a wavelength of 270 nm and impurities quantitated relative to an external standard of Enzalutamide with no response factors applied.

Stability of formulations was monitored by exposing them to elevated temperature (50° C., 30% relative humidity) in an open glass vial for 14 days. After storage, formulations were analyzed and the amounts of degradation products measured by HPLC. The extent of degradation was determined by subtracting the total amount of degradation products of a non-stressed (control) sample from the total amount of degradation of a stressed sample.

### Reference Examples 1 and 2: Currently Marketed Product with and without Antioxidants

Currently marketed product Xtandi (Reference example 1) is formulated as saturated solution of Enzalutamide dissolved in surfactant caprylocaproyl polyoxylglycerides (Labrasol®) with added antioxidants (BHA and BHT), filled into soft gelatin capsules.

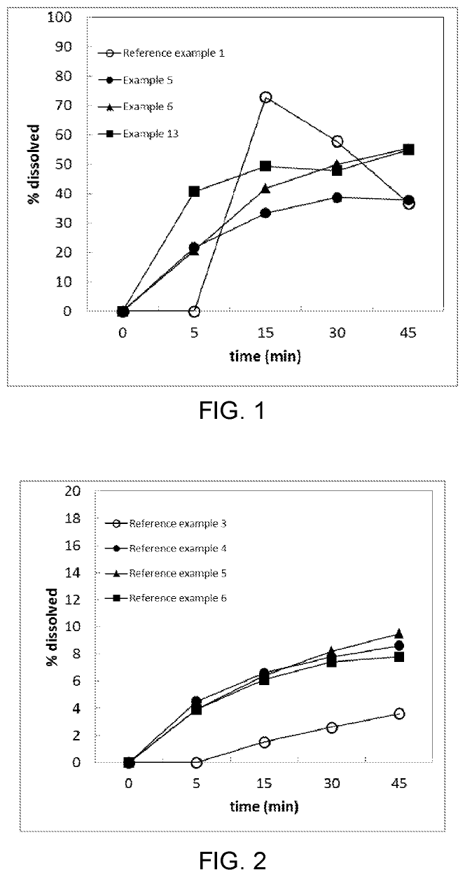

Dissolution of Enzalutamide from Xtandi is displayed in FIG. 1. In the diagram there is a lag of about 5-10 minutes needed for soft gelatin capsule to disintegrate. Enzalutamide concentration decreases significantly at times >15 min due to precipitation.

Key performance attributes of Reference Examples 1 and 2 are collected in Table 1. Reference Example 1 is characterized by fast dissolution and good stability, however at the expense of large dosage unit size and high content of ingredients that increase bio-burden to patients (surface active molecules, antioxidants). The addition of antioxidants is necessary, since Enzalutamide solution in Labrasol® alone (Reference example 2) is very unstable.

TABLE 1
Performance attributes of Reference Examples 1 and 2

| | % dissolved | 14-day degradation | Volume of | Surface active | |
|---|---|---|---|---|---|
| Reference | at 45 min in | products increase at | single dosage | ingredient per 40 | Contains |
| Example: | FaSSIF pH 6.5 | 50° C./30 RH (%) | unit (cm3) | mg of API (mg) | antioxidants |
| 1) Xtandi | 59.3 | No increase | 1.3 | 900 | YES |
| 2) Enzalutamide | Expected to | 14.55 | 1.3 | 900 | NO |
| in Labrasol | be same as | | | | |
| | Xtandi | | | | |

### Reference Example 3: Crystalline Enzalutamide in a Generic Formulation with Filler

Opposed to liquid formulation of Xtandi (Reference Example 1), an entirely solid formulation composed of crystalline Enzalutamide and lactose in a ratio of 1:20 has been prepared (see Table below). This formulation is characterized by slow dissolution compared to Xtandi as can be observed by the comparison between FIG. 2 and FIG. 1. Only 3.6% of the dose dissolved in 45 minutes in 500 ml of FaSSIF pH 6.5.

### Reference Example 4: Crystalline Enzalutamide with Surfactant

An entirely solid formulation composed of crystalline Enzalutamide and sodium lauryl sulphate (SLS) in a ratio of 1:5 has been prepared (see Table below). This formulation is characterized by slow dissolution compared to Xtandi as can be observed by the comparison between FIG. 2 and FIG. 1. Only 8.6% of the dose dissolved in 45 minutes in 500 ml of FaSSIF pH 6.5.

### Reference Examples 5 and 6: Crystalline Enzalutamide with Reduced Particle Size with Addition of Suspension Stabilizers

Reference Examples 5 and 6 illustrate insufficient effects on Enzalutamide dissolution despite of particle size reduction by wet milling in presence of suspension stabilizer. Ingredients are shown in the Table below. As suspension stabilizer, a surfactant is used in Reference Example 5 and a polymer in Reference Example 6. Sucrose was added to the suspension, which was then freeze dried and filled into capsules. Nanosuspensions were produced as follows. Stabilizer was dissolved in water, Enzalutamide (with particle size d05=35 μm) was added and homogeneously suspended in the solution and zirconium oxide milling balls were added. Both suspensions were milled in a planetary ball mill at 500 rpm for 3 hours. Resulting nanosuspensions were analysed with laser diffraction method. Median particle size (d05) was determined as 0.37 μm for Reference Example 4 and 0.12 μm for Reference Example 5, which is considered to be close to practical limit of wet milling.

Table summarizing compositions of Reference Examples 3 to 6:

| | | Ref Ex. 3 | Ref. Ex. 4 | Ref. Ex. 5 | Ref. Ex. 6 |
|---|---|---|---|---|---|
| ingredient | function | mg/unit | mg/ unit | mg/unit | mg/unit |
| Enzalutamide | active | 40.00 | 40.00 | 40.00 | 40.00 |
| | ingredient | | | | |
| Lactose | filler | 760.00 | / | / | / |
| Sucrose | filler | / | / | 120.00 | 120.00 |
| Sodium | surfactant | / | 200.00 | 20.00 | / |
| Lauryl | | | | | |
| Sulphate | | | | | |
| HPMC | polymer | / | / | / | 20.00 |
| Total | | 800.00 | 240.00 | 180.00 | 180.00 |

Sizing of particles improves dissolution notably as evident from table.

| Example: | % dissolved at 45 min in FaSSIF pH 6.5 |
|---|---|
| Ref. Example 3 | 3.6 |
| Ref. Example 4 | 8.6 |
| Ref. Example 5 | 7.8 |
| Ref. Example 6 | 9.5 |

In FIG. 2 the results on dissolution of Enzalutamide from formulation of Reference Examples 3, 4, 5 and 6 are displayed. Reference Example 3 is a mixture of lactose and crystalline Enzalutamide with particle size parameter d05 below 40 um. Reference Example 4 is a mixture of API from reference example 3 and sodium lauryl sulphate. Reference Examples 5 and 6 comprise Enzalutamide with particle size reduced down d05 to about 0.1 um, which is about the practical limit of API wet milling. Formulations of Reference Examples 5 and 6 in addition contain surfactant and polymer respectively, which ensures stabilization of the suspension of micronized API particles.

From results of dissolution displayed on FIG. 2 it is obvious that there is an increase of dissolution rate for the formulations containing micronized API and formulation containing considerable amount of surfactant. Nevertheless, all three improved formulations (Reference Examples 4, 5 and 6) fail to meet the dissolution criterion (NLT 35% in bio-relevant medium) by about factor of 4, even though they contain surfactants and precipitation inhibitors. From these results, it becomes apparent that reduction of crystalline API particle size to a practical limit of industrially applicable wet milling gives insufficient enhancement of dissolution, despite it was used in combination with surfactant or polymer in a role of suspension stabilizer and/or precipitation inhibitor.

### Reference Example 7: ARN-509

40.00 mg of ARN-509 was filled into hard gelatin capsule. 12.2% of the dose dissolved in 45 minutes in 500 ml of FaSSIF pH 6.5.

### Example 1a: Manufacturing Procedure of 10% Enzalutamide Adsorbate on Syloid

1 g of Enzalutamide was dissolved in 25 ml of dichloromethane. 10 g of dried porous silicon dioxide Syloid AL1 (originally having a BET specific surface area of 750 m²/g) was added to the solution and stirred. Slowly 100 ml of n-hexane was added to the solution and stirred. The solvents were slowly removed under reduced pressure over a period of one hour. The solvents were further removed at 50° C. and 10 mbar for 8 hours.

### Example 1b: Manufacturing Procedure of 5% Enzalutamide Adsorbate on Syloid

0.5 g of Enzalutamide was dissolved in 25 ml of dichloromethane. 10 g of dried porous silicon dioxide Syloid AL1 was added to the solution and stirred. Slowly 100 ml of n-hexane was added to the solution and stirred. The solvents were slowly removed under reduced pressure over a period of one hour. The solvents were further removed at 50° C. and 10 mbar for 8 hours.

### Example 1c: Manufacturing Procedure of 20% Enzalutamide Adsorbate on Syloid

2 g of Enzalutamide was dissolved in 25 ml of dichloromethane. 10 g of dried porous silicon dioxide Syloid AL1 was added to the solution and stirred. Slowly 100 ml of n-hexane was added to the solution and stirred. The solvents were slowly removed under reduced pressure over a period of one hour. The solvents were further removed at 50° C. and 10 mbar for 8 hours.

### Example 1d: Manufacturing Procedure of 10% Enzalutamide Adsorbate on Neusilin

1 g of Enzalutamide was dissolved in 25 ml of dichloromethane. 10 g of dried Neusilin was added to the solution and stirred. Slowly 100 ml of n-hexane was added to the solution and stirred. The solvents were slowly removed under reduced pressure over a period of one hour. The solvents were further removed at 50° C. and 10 mbar for 8 hours.

### Example 2a: Manufacturing Procedure of 10% ARN-509 Adsorbate on Syloid

1 g of ARN-509 was dissolved in 25 ml of dichloromethane. 10 g of dried porous silicon dioxide Syloid AL1 was added to the solution and stirred. Slowly 100 ml of n-hexane was added to the solution and stirred. The solvents were slowly removed under reduced pressure over a period of one hour. The solvents were further removed at 50° C. and 10 mbar for 8 hours.

### Example 2b: Manufacturing Procedure of 10% ARN-509 Adsorbate on Neusilin

1 g of ARN-509 was dissolved in 25 ml of dichloromethane. 10 g of dried Neusilin was added to the solution and stirred. Slowly 100 ml of n-hexane was added to the solution and stirred. The solvents were slowly removed under reduced pressure over a period of one hour. The solvents were further removed at 50° C. and 10 mbar for 8 hours.

### Examples 3 and 4 and Reference Example 8: Final Dosage Forms of Enzalutamide Adsorbate and Surfactant

Different compositions were prepared as shown in the ingredient list below, with 5% Enzalutamide adsorbate prepared according to Example 1b and with different ingredients that enhance wetting: lactose as hydrophilic substance, and/or SLS (Sodium Lauryl Sulphate) as surface active substance (solid surfactant).

Samples were produced by mixing together Enzalutamide adsorbate, lactose and/or SLS with pestle and mortar. The resulting granulate was filled into hard gelatin capsules or compressed into tablets, corresponding to 40 mg of Enzalutamide per capsule/tablet.

Table summarizing compositions of Examples 3 and 4 and Reference Example 8:

| | | | | Reference |
|---|---|---|---|---|
| | | Example 3 | Example 4 | Example 8 |
| ingredient | function | mg/unit | mg/unit | mg/unit |
| Enzalutamide | active | 800.00 | 800.00 | 800.00 |
| adsorbate (5%) | ingredient | | | |
| Lactose | filler | / | 100.00 | 200.00 |
| Sodium Lauryl Sulphate | surfactant | 200.00 | 100.00 | / |
| Total | | 1000.00 | 1000.00 | 1000.00 |

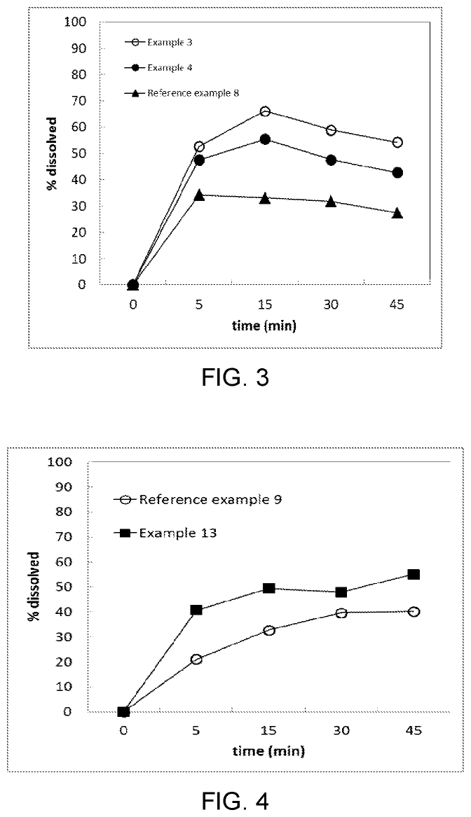

Results are shown in the Table below and in FIG. 3. All samples demonstrate acceptable stability. The use of adsorbates (Examples 3 and 4) significantly improves several-fold the dissolution in comparison to Reference Examples 3-6. The use of surfactant (SLS) resulted in higher dissolution rates compared to use of hydrophilic substance (lactose), though both substances act as enhancers of wetting. In samples with surfactant the threshold dissolution NLT 35% at 45 min is met.

| | % dissolved | Volume of | Surface active | Contains |
|---|---|---|---|---|
| | at 45 min in | single dosage | ingredient per 40 | anti- |
| Example: | FaSSIF pH 6.5 | unit (cm3) | mg of API (mg) | oxidants |
| Example 3 | 54.3 | 0.75 | 200 | NO |
| Example 4 | 42.7 | 0.75 | 100 | NO |
| Reference | 26.4 | 0.75 | 0 | NO |
| Example 8 | | | | |

### Examples 5 and 6: Final Dosage Forms of Enzalutamide Adsorbate and Surfactant

We prepared different compositions with 20% Enzalutamide adsorbate according to Example 1c together with different surface active molecules and polymers, which were Sodium Lauryl Sulphate (SLS) and Polyethylene glycol 6000 (PEG 6000).

Samples were produced by mixing together Enzalutamide adsorbate and other ingredients with pestle and mortar. The resulting granulate was filled into hard gelatin capsules or compressed into tablets, corresponding to 40 mg of Enzalutamide per capsule/tablet.

| | | Example 5 | Example 6 |
|---|---|---|---|
| ingredient | function | mg/unit | mg/unit |
| Enzalutannide | active ingredient | 200.00 | 200.00 |
| adsorbate 20% | | | |
| SLS | surfactant | 20.00 | |
| PEG 6000 | surfactant | / | 40.00 |
| Ac-Di-Sol | disintegrant | 11.00 | / |
| Magnesium stearate | lubricant | 1.10 | / |
| Total | | 232.10 | 240.00 |

All samples demonstrate acceptable stability. Use of highly concentrated adsorbate (20%) gives acceptable dissolution compared to Xtandi (see FIG. 1) even though the amount of surface active ingredient is reduced down to as little as 20.00 mg per single dose of Enzalutamide (40.00 mg). This represents a 44-fold improvement over Xtandi. At such low level the current IIG daily intake limit for SLS (51.7 mg) is entirely met for single (40.00 mg) and double (80.00 mg) dose of Enzalutamide and is close to being met for maximum recommended daily dose of Enzalutamide (160.00 mg). Current IIG daily intake limit for PEG 6000 (375 mg) is fully met in Example 6 even for maximum recommended daily dose of Enzalutamide. Furthermore, all samples demonstrate notable reduction of dosage unit size. At 0.17 cm3, the dosage form size is improved by over 7-fold over Xtandi and makes it possible for the highest recommended daily dose of Enzalutamide (160.00 mg) to be formulated as a single tablet with weight as low as 680 mg.

| | % dissolved | Volume of | Surface active | Contains |
|---|---|---|---|---|
| | at 45 min in | single dosage | ingredient per 40 | anti- |
| Example: | FaSSIF pH 6.5 | unit (cm3) | mg of API (mg) | oxidants |
| Example | 37.9 | 0.19 | 20 | NO |
| 5 | | | | |
| Example | 55.5 | 0.17 | 40 | NO |
| 6 | | | | |

### Examples 7 and 8: Final Dosage Forms of Enzalutamide Adsorbate and Surfactant

Composition was prepared with 10% Enzalutamide adsorbate on Neusilin from Example 1d) and Sodium Lauryl Sulphate (SLS). Samples were produced by mixing together Enzalutamide adsorbate and other ingredients with pestle and mortar. Ac-Di-Sol and Magnesium Stearate were added. The resulting granulate was filled into hard gelatin capsules or compressed into tablets, corresponding to 40 mg of Enzalutamide per capsule/tablet (see ingredient Table below).

| | | Example 7 | Example 8 |
|---|---|---|---|
| ingredient | function | mg/unit | mg/unit |
| Enzalutannide | active ingredient | 400.00 | 400.00 |
| adsorbate 10% | | | |
| SLS | surfactant | 40.00 | 20.00 |
| Ac-Di-Sol | disintegrant | / | 21.00 |
| Magnesium stearate | lubricant | / | 2.10 |
| Total | | 440.00 | 443.10 |

Both examples demonstrate acceptable stability. The threshold dissolution NLT 35% at 45 min is met for both samples even at surface active ingredient content as low 20.00 mg per single dose of Enzalutamide (40.00 mg).

| | % dissolved | Volume of | Surface active | Contains |
|---|---|---|---|---|
| | at 45 min in | single dosage | ingredient per 40 | anti- |
| Example: | FaSSIF pH 6.5 | unit (cm3) | mg of API (mg) | oxidants |
| Example | 65.7 | 0.35 | 40 | NO |
| 7 | | | | |
| Example | 64.5 | 0.35 | 20 | NO |
| 8 | | | | |

### Examples 9 and 10: Final Dosage Forms of ARN-509 Adsorbate and Surfactant

Composition was prepared with 10% ARN-509 adsorbate on Syloid from Example 2a) and Sodium Lauryl Sulphate (SLS). Sample was produced by mixing together ARN-509 adsorbate and other ingredients with pestle and mortar. Ac-Di-Sol and Magnesium Stearate were added. The resulting granulate was filled into hard gelatin capsules or compressed into tablets, corresponding to 40 mg of Enzalutamide per capsule/tablet.

| | | Example 9 | Example 10 |
|---|---|---|---|
| ingredient | function | mg/unit | mg/unit |
| ARN-509 | active ingredient | 400.00 | 400.00 |
| adsorbate 10% | | | |
| SLS | surfactant | 40.00 | 40.00 |
| Ac-Di-Sol | disintegrant | / | 22.00 |
| Magnesium stearate | lubricant | / | 2.20 |
| Total | | 440.00 | 464.10 |

Dissolution of both examples significantly exceeds dissolution of Reference Example 6 (prior art) by 5-7 times. Further, it is highly beneficial by enabling small dosage form volume and low content of surface active substance.

| | % dissolved | Volume of | Surface active | Contains |
|---|---|---|---|---|
| | at 45 min in | single dosage | ingredient per 40 | anti- |
| Example: | FaSSIF pH 6.5 | unit (cm3) | mg of API (mg) | oxidants |
| Example | 77.6 | 0.35 | 40 | NO |
| 9 | | | | |
| Example | 61.9 | 0.35 | 40 | NO |
| 10 | | | | |

### Examples 11 and Reference Example 9: Final Dosage Forms of ARN-509 Adsorbate and Surfactant

Composition was prepared with 10% ARN-509 adsorbate on Neusilin from Example 2b and Sodium Lauryl Sulphate (SLS). Sample was produced by mixing together ARN-509 adsorbate and other ingredients with pestle and mortar. The resulting granulate was filled into hard gelatin capsules or compressed into tablets, corresponding to 40 mg of Enzalutamide per capsule/tablet.

| | | Example 11 | Ref. Example 9 |
|---|---|---|---|
| ingredient | function | mg/unit | mg/unit |
| ARN-509 | active ingredient | 400.00 | 400.00 |
| adsorbate 10% | | | |
| SLS | surfactant | 13.00 | |
| Total | | 413.00 | 400.00 |

Dissolution of both examples exceeds dissolution of Reference Example 6 (pure ARN-509) by 3-4 times and is comparable or better to that of Xtandi at significantly smaller dosage form volume. Dissolution is significantly improved with addition of surface active substance in a quantity as low as 13.00 mg per single dose of ARN-509 (40.00 mg). This represents an almost 70-fold improvement over Xtandi. At such low level the current IIG daily intake limit for SLS (51.7 mg) is met for a daily dose of ARN-509 of 160.00 mg.

| | % dissolved | Volume of | Surface active | Contains |
|---|---|---|---|---|
| | at 45 min in | single dosage | ingredient per 40 | anti- |
| Example: | FaSSIF pH 6.5 | unit (cm3) | mg of API (mg) | oxidants |
| Example 11 | 47.5 | 0.33 | 13 | NO |
| Ref. | 39.8 | 0.33 | 0 | NO |
| Example 9 | | | | |

### Reference Example 10 and Example 12: Final Dosage Forms of Enzalutamide Solid Dispersion

Enzalutamide was fully dissolved in acetone. Hydroxypropyl Methylcellulose (HPMC) and Sodium Lauryl Sulphate (SLS) were added and dispersed. This mixture was poured onto solid carrier, which was microcrystalline cellulose (Avicel) and mixed until granulate was formed. Granulate was then dried for two hours in vacuum dryer at 40° C. Dried granulate was filled into hard gelatin capsules corresponding to 40 mg of Enzalutamide per capsule.

| | | Reference | |
|---|---|---|---|
| | | Example 10 | Example 12 |
| ingredient | function | mg/unit | mg/unit |
| Enzalutamide | active | 40.00 | 40.00 |
| | ingredient | | |
| HPMC | polymer | 40.00 | 40.00 |
| Avicel | filler | 440.00 | 440.00 |
| SLS | surfactant | / | 40.00 |
| Total | | 520.00 | 560.00 |

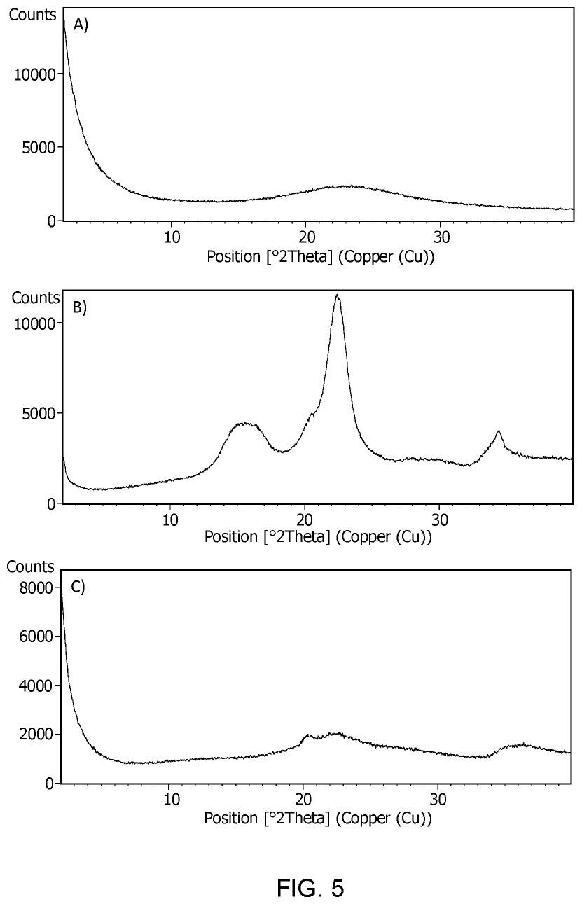

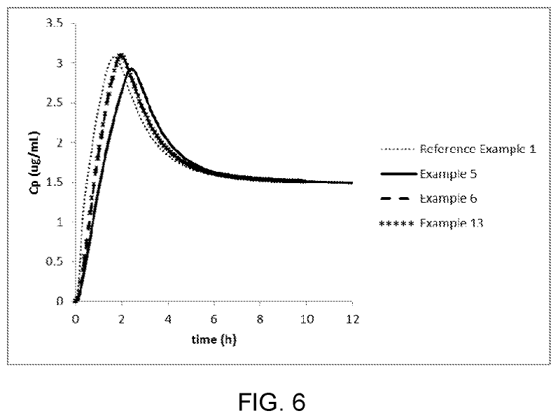

All samples demonstrate acceptable stability. In both Examples Enzalutamide was found to be present only in amorphous form (FIG. 5B). The threshold dissolution NLT 35% at 45 min is met for both examples. Introduction of a surfactant notably improves dissolution. At 45 min the level of dissolution (FIG. 4) of Example 12 is superior to that of Xtandi at 22-times lower content of surfactant.

| | % dissolved | Volume of | Surface active | Contains |
|---|---|---|---|---|
| | at 45 min in | single dosage | ingredient per 40 | anti- |
| Example: | FaSSIF pH 6.5 | unit (cm3) | mg of API (mg) | oxidants |
| Reference | 40.2 | 0.40 | 0 | NO |
| Example 10 | | | | |
| Example 12 | 55.4 | 0.43 | 40 | NO |

### Example 13: Confirmation of Amorphous Form

By way of X-ray diffractogram and as confirmed by the corresponding results shown in FIG. 5A-5C, both the deposition of molecules within the adsorbates (Enzalutamide sample of Example 1a, shown in FIG. 5A, and ARN-509 sample of Example 10, shown in FIG. 5C) and dispersions of molecules within a solid dispersion (Enzalutamide sample of Reference Example 9 shown in FIG. 5B) according to the present invention are such that they prevent recrystallization, i.e. they result in completely amorphous active ingredient.

XRP diffractograms of the tablets according to the present invention only show placebo peaks, thus confirming that only amorphous Enzalutamide or ARN-509 is present in the samples.

### Example 14: Precipitation Inhibition

A further experiment was designed to further investigated whether it is possible, and if so which type of substance, may exert effects on stabilizing the compound of formula 1, once it is in liquid dissolved state.

To this end, introduction of surface active agents (surfactants) or a suitable polymer in the composition was investigated. As illustrated in the Table below, time dependent decrease of Enzalutamide concentration in pH 6.8 phosphate buffer due to precipitation of Enzalutamide from three different liquid formulations is displayed.

In the first reference test formulation, 12 mg of Enzalutamide was completely dissolved in 0.27 ml of Labrasol. In second reference test formulations 12 mg of Enzalutamide is completely dissolved in 0.27 ml of Tween 80. In third reference test formulation, 12 mg of Enzalutamide is completely dissolved in 0.27 ml of Tween 80 and 0.15 mg of HPMC has been added. It should be noted that solubility of Enzalutamide is about the same in Tween and in Labrasol (about 36 mg of Enzalutamide per 1 ml of either Tween 80 or Labrasol). The three test formulations were dissolved in 15 ml of pH 6.8 phosphate buffer and Enzalutamide concentration was measured at time points from 10 to 360 minutes.

By comparing Enzalutamide concentrations at individual timepoints for three reference test formulations (see Table below) it can be seen that Tween 80 inhibits the precipitation of Enzalutamide much better than Labrasol. Also it can be seen that addition of HPMC, in an amount as small as 1% with respect to Enzalutamide, stably maintains a significantly higher concentration of Enzalutamide dissolved in the medium; in comparison this is an increase by an additional factor of 2 or more. In particular, addition of hydrophilic polymers, such as HPMC, results in significantly improved precipitation inhibition over Labrasol in Xtandi.

Hence, the present experimental test demonstrates that introduction of an excipient that inhibits precipitation of a compound of formula 1, for example a correspondingly selected and suitable hydrophilic, water soluble polymer, improves the solid pharmaceutical composition of the present invention in terms of improved dissolution performance (dissolution stability with reduced or without compound precipitation).

| | % of Enzalutamide dissolved | | |
|---|---|---|---|
| Time (min) | Labrasol | Tween 80 | Tween 80 + HPMC |
| 10 | 0.49 | 4.72 | 11.04 |
| 20 | 0.52 | 4.12 | 8.02 |
| 60 | 0.08 | 3.78 | 8.96 |
| 120 | 0.06 | 3.20 | 6.74 |
| 240 | 0.03 | 3.22 | 8.28 |
| 360 | 0.06 | 3.30 | 7.98 |

1. A solid pharmaceutical composition comprising
(a) ARN-509 denoted by the following formula: 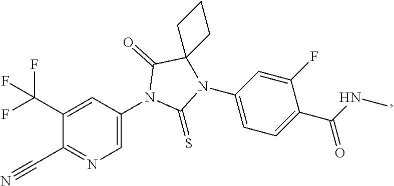 [SMILES: CNC(=O)c1ccc(N2C(=S)N(c3cnc(C#N)c(C(F)(F)F)c3)C(=O)C23CCC3)cc1F]
(b) a carrier, and
(c) a surfactant
wherein the ARN-509 is mainly amorphous and components (a) and (b) are combined in the form of a solid adsorbate of the ARN-509 being adsorbed on the surface of a carrier, or components (a) and (b) are combined in the form of a solid dispersion of the ARN-509 with a polymer.

2-3. (canceled)

4. The solid pharmaceutical composition according to claim 1, wherein the amount of surfactant is limited by a weight ratio of the surfactant to the ARN-509 being not higher than 10:1.

5. The solid pharmaceutical composition according to claim 1, having a dissolution ratio of the ARN-509 of not less than (NLT) 35%, when the pharmaceutical composition is subjected to a dissolution test in fasted state simulated intestinal fluid (FaSSIF) pH 6.5 medium at 45 minutes and at 100 rpm in USP Apparatus 2 (paddle method).

6. The solid pharmaceutical composition according to claim 1, wherein the amount of the ARN-509 in the entire composition is greater than 5%.

7. The solid pharmaceutical composition according to claim 1, wherein the surfactant is selected from the group consisting of sodium lauryl sulphate; polyethylene glycol having molecular weight in the range of about 2000 to 10000; Polysorbates; fatty acid esters; esters of glycerol and fatty acids; esters of polyethylene glycol and fatty acids, and castor oil ethoxylate.

8-9. (canceled)

10. The solid pharmaceutical composition according to claim 9, wherein the solid dispersion or solid solution is formed with a hydrophilic, water soluble polymer.

11. The solid pharmaceutical composition according to claim 1, which is in the form of a hard gelatine capsule or a tablet.

12. A process for the preparation of a solid pharmaceutical composition according to claim 1 comprising one or more step(s) of mixing said ARN-509, said carrier and said surfactant.

13. The process according to claim 12, wherein the one or more step(s) of mixing comprises:
a) providing a solution of the ARN-509 in a solvent or mixture of solvents dissolving said ARN-509;
b) mixing a solution of a) with a solid adsorbate carrier;
c) drying the mixture of b) to thereby yield a solid adsorbate of said the ARN-509 being adsorbed on the surface of said solid adsorbate carrier; and
d) optionally carrying out further processing steps selected from granulation, compression, tableting, pelletisation, capsulation, and coating,
wherein said surfactant is added in any one of steps a) to d).

14. The process according to claim 12, wherein the one or more step(s) of mixing comprises:
a′) providing a solution of the ARN-509 in a solvent or mixture of solvents dissolving said compound, and adding a polymer to obtain a solution or dispersion additionally containing the polymer as a carrier;
b′) optionally mixing the solution or dispersion of a′) with one or more further excipients,
c) drying the mixture of a′) or b′) to yield a composition comprising a solid dispersion or solid solution of said compound of formula 1 with said polymer; and
d) optionally carrying out further processing steps selected from granulation, compression, tableting, pelletisation, capsulation, and coating,
wherein said surfactant is added in any one of steps a′) to d).

15. Solid pharmaceutical composition according to claim 1 for use in the treatment of prostate cancer.
